(19) United States
(12) Patent Application Publication
(10) Pub. No.: US 2022/0071306 A1
(43) Pub. Date: Mar. 10, 2022

(54) DOSAGE TESTING

(71) Applicant: Radient Technologies Innovations Inc., Edmonton (CA)

(72) Inventors: Michael Cabigon, Demonton, CA (US); Jim Seethram, Edmonton (CA); Steven Splinter, Edmonton (CA); Denis Taschuk, Edmonton (CA)

(21) Appl. No.: 17/239,769

(22) Filed: Apr. 26, 2021

Related U.S. Application Data
(63) Continuation of application No. PCT/IB2019/058918, filed on Oct. 18, 2019.
(60) Provisional application No. 62/750,134, filed on Oct. 24, 2018.

(51) Int. Cl. A24F 40/80; A24F 40/10; G01N 1/44; G01N 33/00

(52) U.S. Cl. CPC A24F 40/80; G01N 33/0047; G01N 1/44; A24F 40/10

(57) ABSTRACT

Methods and systems for testing Cannabis vaping liquid for liquid vaporizers and vaporizer devices to ensure compliance with product quality are disclosed. The methods and systems allow Cannabis extractors to test Cannabis vaping liquids produced with the Cannabis extract by using a universal attachment couplable with a vaporizer device at various settings of the vaporizer device. The test results can be stored in the cloud accessible by Cannabis extractors, Cannabis-infused product producers, vaporizer device manufacturers, and end users so that the quality data of the vaping liquid and optimum vaporizer settings are easily determined.

### CROSS-REFERENCE TO RELATED APPLICATIONS

The present patent application is a continuation of International Application No. PCT/IB2019/058918 filed Oct. 18, 2019, which claims the priority benefit of U.S. provisional patent application No. 62/750,134 filed Oct. 24, 2018, the disclosures of which are incorporated by reference herein.

### BACKGROUND OF THE INVENTION

### 1. Field of the Disclosure

The present disclosure is generally related to a Cannabis concentrate use quality control. More specifically, the present disclosure relates to a method for providing a Cannabis active ingredient, analyzing and providing the result of a dosage testing device and testing procedure.

### 2. Description of Related Art

Cannabis is a genus belonging to the family cannabaceae. There are three common species of Cannabis including Cannabis stavia, Cannabis indica, and Cannabis ruderalis. The genus cannabaceae is indigenous to Central Asia and the Indian subcontinent and has a long history of being used for medicinal, therapeutic, and recreational purposes. For example, Cannabis is known to be capable of relieving nausea (such as that accompanying chemotherapy), pain, vomiting, spasticity in multiple sclerosis, and increase hunger in anorexia. The term Cannabis as used herein can refer to a “Cannabis biomass” which can encompass the Cannabis sativa plant and variants thereof, including subspecies sativa, indica and ruderalis, Cannabis cultivars, and Cannabis chemovars (varieties characterised by chemical composition). The term “Cannabis biomass” is to be interpreted accordingly as encompassing plant material derived from one or more Cannabis plants. Such Cannabis biomasses can naturally contain different amounts of the individual cannabinoids.

Cannabis biomasses contain a unique class of terpeno-phenolic compounds known as cannabinoids, or phytocannabinoids. The principle cannabinoids present in a Cannabis biomass can include Delta-9-tetrahydrocannabinolic acid (THCA) and cannabidiolic acid (CBDA). THCA does not include psychoactive properties on it's own, but when decarboxylated THCA becomes Delta-9-tetrahydrocannabinol (THC), which is a potent psychoactive cannabinoid. CBDA can be decarboxylated into cannabidiol (CBD), which is a major cannabinoid substituent in hemp Cannabis. CBD is a non-psychoactive cannabinoid and is widely known to have therapeutic potential for a variety of medical conditions including, but not limited to, those described above.

Historical delivery methods of cannabinoids have included combustion (such as smoking) of the dried Cannabis plant material, or biomass. However, smoking can result in adverse effects on a user's respiratory system due to the production of potentially toxic substances. Moreover, smoking is an inefficient mechanism which delivers a variable mixture of both active and inactive substances, many of which may be undesirable. Common alternative delivery methods, including but not limited to, ingestion, topical and oral mucosal, typically require an extraction process to be performed on the Cannabis biomass to remove and concentrate the desired components. These extract products are often referred to as Cannabis concentrates, extracts or oils. In at least one example, Cannabis extracts can be formulated into liquids that can be used in a vaporizer. Such liquids are sometimes referred to as Cannabis vaping liquids or “vape oils”. Maintaining quality control for Cannabis substances to be used with vaporizers is important due to a lack of health and safety data for end users in the Cannabis market. Liquid vaporizers, for use with Cannabis vaping liquids , are similar to those used for nicotine, in that there is no combustion of the vaporized oil or liquid. Therefore the delivery of cannabinoids avoids the hazards of smoking by combusting Cannabis (such as the formation of toxic pyrolytic byproducts). However, every vaporizer can deliver a different experience, based on a combination of temperature at which the vaporizer operates and the Cannabis vaping liquid used. As such, it is difficult for end users to determine the exact potency and effects of the vape oil or vape liquid they have purchased.

### SUMMARY OF THE CLAIMED INVENTION

Examples of the present disclosure provide systems and methods for evaluating the quality of a Cannabis vaping liquid and the optimum settings of a vaporizer device to be used with the Cannabis vaping liquids. In particular, a system for testing various vaporizer settings with various Cannabis-infused vaping liquids can include a vaporizer, an attachment device, and an analysis network communicatively coupled with one another via a communication network. The analysis network can analyze data obtained by the attachment device and store the analysis of each vaporizer and Cannabis-infused vaping liquid for later retrieval. The analysis can be used to determine the optimum vaporizer settings for a vaporizer when a specific Cannabis-infused vaping liquid is used.

In addition to improving end user knowledge of the Cannabis-infused vaping liquid they are using, such systems and methods can further provide extractors and Cannabis-infused product producers with a way to ensure quality control of their end products. The systems can also allow end users to better understand the optimum settings of the vaporizers and the health effects of the vaporizer and Cannabis-infused vaping liquid they are using.

### BRIEF DESCRIPTIONS OF THE DRAWINGS

FIG. 1 illustrates an exemplary network environment in which a Cannabis dosage testing system may be implemented.

FIG. 2 illustrates exemplary graphs of draw and release over time.

FIG. 3 illustrates an exemplary attachment device operable with the Cannabis dosage testing system.

FIG. 4 illustrates an exemplary method for obtaining data relating to dosage.

FIG. 5 illustrates an exemplary method for analyzing optimal settings for a vaporizer.

### DETAILED DESCRIPTION

Cannabis vaping liquids, sometimes called “vape oils” are currently not FDA regulated and therefore the concentration, or potency, of the oils are not evaluated for quality control. As such, the end user needs an easy way to check the potency and other elements within the Cannabis vaping liquid. Major risks can be associated with Cannabis vaping if the electronic device, or vaporizer, is misused. This can be exacerbated by poor quality control of the chemical compositions of liquid refills which are vaporized.

This present disclosure provides a method and system allowing end users access to quality parameters of the Cannabis vaping liquids to be used with a vaporizer device. Such systems can also be useful for Cannabis extractors and formulators of Cannabis vaping liquids because they will be able to test the final liquid products in the actual vaping devices that end users will use the products with, and provide clarity and information to end users.

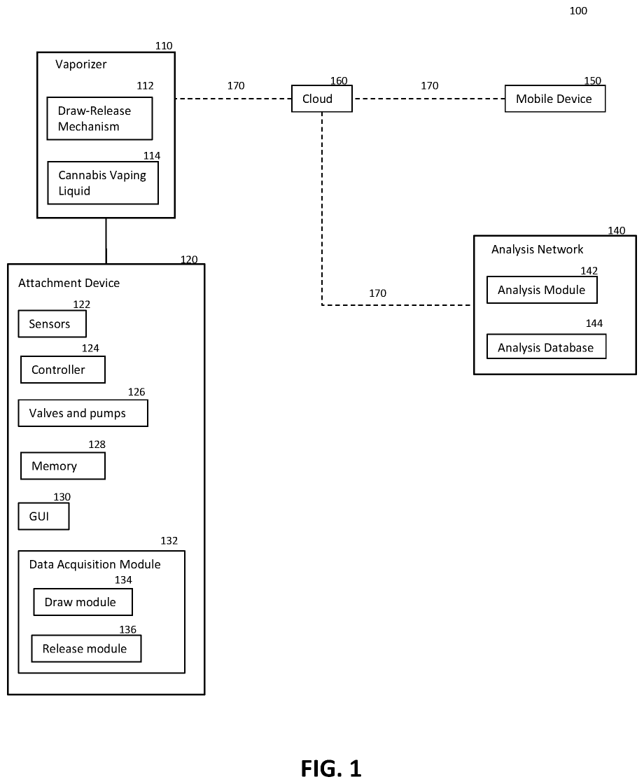

A Cannabis dosage testing system 100 operable to evaluate the potency and composition of one or more Cannabis vaping liquids is described with respect to FIG. 1, which illustrates an exemplary network environment in which a Cannabis dosage testing system may be implemented. The system 100 comprises at least a vaporizer device 110 which can be used with Cannabis-infused vaping liquids 114. The vaporizer device 110 of system 100 can include any standard vaporizer operable to deliver an active ingredient, such as those which are known in the art. Certain vaporizers known in the art can be designed specifically for the use of Cannabis-infused vape liquids and vape oils. In the alternative, certain can be operable to vaporize ground, dried Cannabis biomasses and herbal oils and liquids are known in the art. The vaporizer device 110 of the present system 100 can be a vaporizer operable to vaporize a dried Cannabis biomass or a Cannabis vaping liquid.

The vaporizer 110 can further include a draw-release mechanism 112, the draw-release mechanism 112 can be activated by either an attachment device 120 or by a user. The system 100 is operable to analyze the potency and composition of Cannabis oil or vaping liquids 114 which may contain high concentrations of cannabinoids including, but not limited to, THC and CBD. In addition to cannabinoids, the system 100 can also be used to determine which solvents or diluents are present in the vaping liquid. The cannabinoids can be extracted from Cannabis plant material using solvents such as butane, ethanol, and supercritical CO2. The system 100 can further comprise an attachment device 120 couplable with the vaporizer 110. The attachment device can have one or more sensors 122, a controller 124, a plurality of valves and pumps 126, a memory 128, a graphical user interface (GUI) 130 embedded thereon and a Data Acquisition Module 132. The vaporizer 110 and the attachment device 120 can be communicable with an analysis network 140 via a communications network 170 such that the attachment device 120 can activate the draw-release mechanism 112 of the vaporizer 110 to vaporize the Cannabis vaping liquid 114. The one or more sensors 122 of the attachment device 120 can be operable to measure various different parameters of the vaporized Cannabis liquid, and transmit the measurements to the analysis network 140 via the communication network 170 for analysis. Once the vaporized Cannabis liquids are analyzed, the information calculated by the analysis network 140 can be transmitted back to the attachment device 120 via the communication network 170 and the attachment device 120 can show the end user the information on the GUI 130 of the attachment device 120.

In at least one example, the attachment device 120 can have an inflatable connector operable to put pressure on a button capable of activating the draw-release mechanism 112 of the vaporizer 110. In an alternative example, the attachment device 120 can connect to the vaporizer 110 through a USB port and is operable to control the draw-release mechanism 112 thereby. The attachment device 120 can be operable to test various draw and release times (t1, t2, t3) and the pulse (amount of draw) for the vaporizer 110. Between each actuated draw, the attachment device 120 can provide a time activated. The analysis network 140 of the system 100 further comprises a plurality of sensors operable to measure different parameters of the vaporized Cannabis liquid as the Cannabis vaping liquid vapor passes through the attachment device 120. At least a portion of the vaporized Cannabis liquid can be captured in a draw chamber of a data acquisition module 132 and a portion of the vaporized Cannabis liquid can be captured in a release chamber. In at least one example, one or more of the sensors 122 can be placed within the draw chamber. The sensor can include, but is not limited to, a sensor operable to detect volatile organic compounds (VOCs) including but not limited to a Sensirion Multi-Pixel Gas Sensor SGP. Additionally, one or more of the sensors can be placed in the release chamber so that the user knows how the amount of VOCs that were absorbed during the inhalation, or puff.

Parameters which are generally tested in e-liquids for e-cigarettes include tobacco, nicotine, flavor, strength, and combinations thereof. The attachment device 120 of the system 100 described herein can further comprise a plurality of valves and pumps 126 operable to allow the vaporized Cannabis liquid to enter the draw chamber of the attachment device 120, maintain the vaporized liquid for a period of time, allow the vaporized liquid to transfer to the release chamber and let it out after a residence time is reached. The attachment device 120 can further comprise a controller 124 operable to manage the plurality of valves and pumps to draw and release the Cannabis vapor and control the length of the draw time. In at least one example, the plurality of valves and pumps can include, but are not limited to, suction pump, push pump, release pump, draw chamber valve, release chamber valve, and combinations thereof.

In at least one example, the attachment device 120 can be operable to control the vaporizer 110, through a USB connection or any other suitable connection, and override all vaporizer 110 settings. As such, the controller 124 of the attachment device 120 can activate the draw of vaporized Cannabis liquid using a draw module 134 and stops the draw when a release module 136 indicates a time out. Additionally, the controller 124 can be operable to store the collected sensor information in a memory 128 of the attachment device and transmit the sensor information to an analysis module 142 of the analysis network 140. The sensor information can then be analyzed at the analysis network 140 and the analysis can be transmitted back to the attachment device 120. The data received from the analysis module 142 can then be displayed for the end user by means of a GUI 130 of the attachment device 120.

The system 100 described herein can further comprise a personal computing device, or mobile device, 150 operable to both accept inputs from users and provide outputs to the users. In at least one example, a user can interact with an interface(s) using one or more user-interactive objects and devices, such as a mobile device 150. The user-interactive objects and devices can include, but are not limited to, user input buttons, switches, knobs, levers, keys, trackballs, touchpads, cameras, microphones, motion sensors, heat sensors, inertial sensors, touch sensors, or a combination of the above. Further, the user-interface(s) may be implemented as a Command Line Interface (CLI), a Graphical User Interface (GUI), a voice interface, or a web-based user-interface operable to show the Cannabis oil or liquid vapor parameters received from the analysis network 140 to the user. In a preferred embodiment, as discussed above, the GUI 130 of the attachment device can also be used to provide the output from the analysis network 140 to the end user.

The system 100 further comprises a communication network 170 capable of providing a connection between the vaporizer 110, the attachment device 120, the analysis network 140, and the mobile device 150. The communications network 170 as described herein may be wired and/or a wireless network. If the communications network 170 is wireless, the communication network 170 may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network 170 may allow ubiquitous access to shared pools of configurable system resources and higher-level services that can be rapidly provisioned with minimal management effort, often over Internet and relies on sharing of resources to achieve coherence and economies of scale, like a public utility, while third-party clouds enable organizations to focus on their core businesses instead of expending resources on computer infrastructure and maintenance.

The system 100 described with respect to FIG. 1 can be used for executing various methods. While several exemplary methods are described herein they are provided for the purposes of this disclosure and are not to be considered limiting. In at least one example, the attachment device 120 can be a testing device. The system 100 described above is further operable to test various Cannabis vaping liquids, several types of vaping liquid can benefit from the type of testing described herein and performed by the system 100. An exemplary method for using a testing device with the system 100 of FIG. 1, a vaping liquid can be tested by loading the Cannabis vaping liquid into a generic vaporizer 110, the vaporizer 110 can then be fitted on a testing device. The testing device can be similar to the attachment device 120 as shown in FIG. 1.

The vaporizer can then be is activated for the lowest temperature and the lowest activation time, such that the shortest puff is simulated. The system 100 as described above can then be used to analyze the vaporized Cannabis liquid via the analysis network 140, the analysis results can then be stored for later retrieval. The process can be repeated and activation time increased in incremental intervals (such as one second intervals). The VOCs emitted through the vaporization process are tested at each increment and the information is stored.

Next, the temperature can be increased in incremental intervals (such as one degree Celsius) while the activation time is increased incrementally (for example, from the shortest puff to longest puff). The process can be repeated for the different temperature levels and the whole process can be repeated for any vaping liquid. Additionally, the testing process can be repeated using any vaporizer device. For example, the testing process can be performed on vaporizer devices available on the market.

In at least one example, the system 100 of FIG. 1 can further comprise a software system, such as the data acquisition module 132, operable to open a suction pump and draw a vapor in from the vaporizer 110 into the draw chamber via the draw module 134 and can close the valve once the draw time is reached. The software system can then be operable to close the valve, via the release module 136, after the vapor has crossed from the draw chamber into the release chamber, open the push pump and once the release time is reached, open the valve to draw the Cannabis vape oil vapor out of the release chamber into the air, via the release module 136.

The analysis network 140 can receive information from the plurality of sensors 122 through the controller 124 in the attachment device 120. The analysis network 132 can then store the data into the analysis database 144 for later retrieval. This data can include, but is not limited to, cannabinoid content, solvent content, diluent content, and combinations thereof. The analysis network 140 can be operable to calculate, by means of the analysis module 142, the different parameters related to the Cannabis vape oil vapor, store those parameters in the analysis database 144, and send the parameter data back to the attachment device 120. The analysis database 140 can also be operable to store data received from the sensors 122 in the attachment device 120 and quality parameters of the Cannabis vaping liquid vapor in the analysis database 144. The system 100 can further comprise a mobile device 150, including, but not limited to, a laptop, a cellphone, a computer, or any other suitable computing device, which the end user of the vaporizer and vaping liquid can use to access the analysis database 144 in the analysis network 140. In at least one example, a user may access the analysis network 140 by logging in to an account accessible via a mobile device 150. The user can send a request to the analysis network 140 for information relating to the settings for a vaporizer device 110. Furthermore, the analysis network 140 can track optimal use parameters for each vaporizer tested and can be operable to estimate what optimal parameters may be for a vaporizer and vaping liquid pairing not previously tested at element 130.

A user can access the analysis network 140 via the mobile device 150 to retrieve information relating to a vaporizer device and liquid. For example, if the user has requested information relating to a vaping liquid that has been tested, the analysis network 140 can transmit the information to the mobile device 150 for the user's review. Additionally, the mobile device 150 can be operable to provide links to health information related to the products they have searched including, but not limited to government regulations and medical articles. In the alternative, if the request is for a vaporizer device which has not been previously tested, an estimate module can be operable to compare data relating to actual tested devices and vaping liquid pairings and to estimate the optimal settings for that untested vaporizer device and liquid combination, if the liquid information is provided by the user. The analysis module 140 can further calculate the different parameters of the Cannabis vaping liquid vapor.

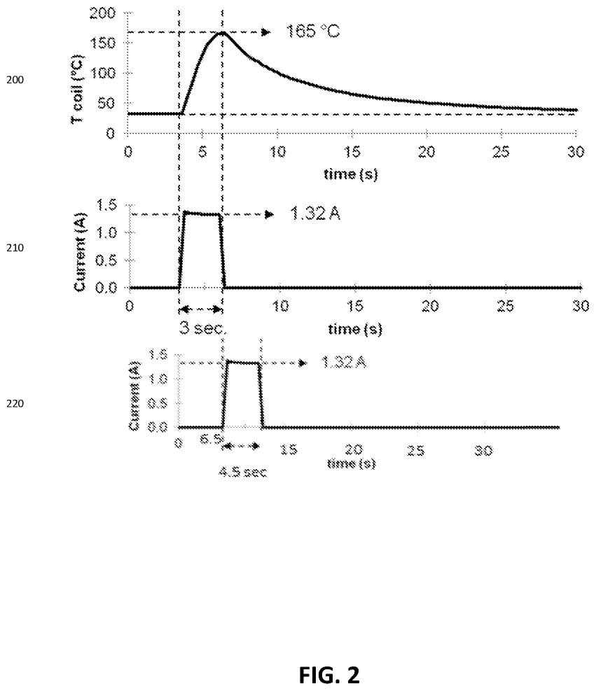

FIG. 2 illustrates exemplary graphs of draw and release over time. Such graphs can be used to analyze the testing device and testing liquids as described above. As described above, a controller of the attachment device can be used to activate a draw cycle using a suction pump. The amount of time which the attachment device draws vaporized liquid from corresponds to the draw cycle of the vaporizer. FIG. 2 illustrates an exemplary draw cycle lasting for three seconds. Specifically, graph 200 illustrates the duration of the draw cycle, for the present example three seconds, and the temperature the coils of the vaporizer reaches during the draw cycle. Graph 210 illustrates the current running through the vaporizer during the same three second draw cycle. Once the vaporized Cannabis liquid is in the draw chamber of the attachment device, the sensors in the draw chamber (described above) can take the desired measurements. After the measurements have been taken, the controller of the attachment device can initiate a release cycle by transferring the vapor from the draw chamber to a release chamber, and releasing the vapor to the exterior of the attachment device. Graph 220 of FIG. 2 illustrates the release time, four and a half seconds, and the current of the attachment device during the release. The graphs illustrated in FIG. 2 can be recorded, along with the sensor analysis data, and stored in the analysis database 114 of the analysis network 140, described with respect to FIG. 1.

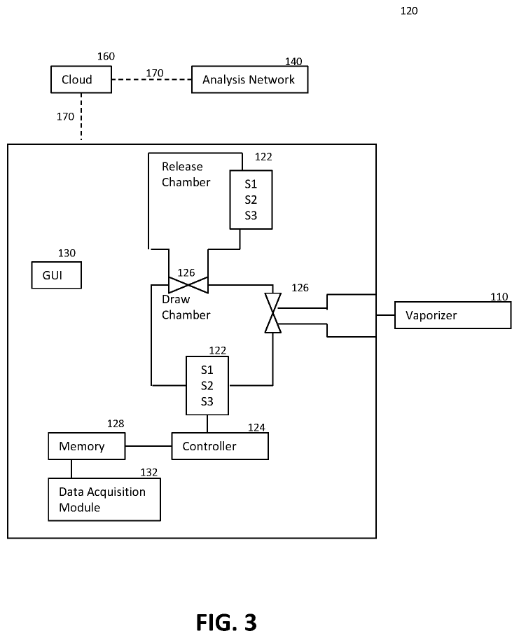

The functioning of the attachment device 140 described above, is explained with reference to FIG. 3, which illustrates an exemplary attachment device operable with the Cannabis dosage testing system. One skilled in the art will appreciate that, the diagram of the attachment device 140 illustrates in FIG. 3 is merely exemplary and that changes can be made to the attachment device 140 without departing from the scope of the present disclosure. FIG. 3 illustrates a detailed diagram of the attachment device. The attachment device 120 illustrated is operable to couple a vaporizer 110 and can include a plurality of sensors (S1, S2, S3 ), a controller, a plurality of valves and pumps 126, a memory, and a GUI. The attachment device can further be coupled to the analysis network 140 described above via a communications network 170. As described above, the attachment device 120 is operable to activate the vaporizer 110 to draw vaporized Cannabis oil or liquid into the attachment device, the sensors (S1, S2, S3) of the attachment device 120 can then be used to measure different parameters of the vaporized Cannabis liquid and send the measurements to the analysis network 140 for a detailed analysis. The attachment device 120 can also be operable to receive the information relating to the detailed analysis performed by the analysis network 140 and display the information to the end user via a GUI 130 located on the attachment device 120. The attachment device can be operable to couple the vaporizer with an inflatable connector, which puts pressure on a button that activates the draw in the vaporizer, or a USB port, operable to control draw and release parameters of the vaporizer. In at least one example, the attachment device 120 can be a testing device as described above and operable to test the optimum settings for the vaporizer 110 for different draw and release times (t1, t2, t3 ) and the pulse (amount of draw). For the safety of the end user, in at least one example, the attachment device 120 can require a time out period between draws.

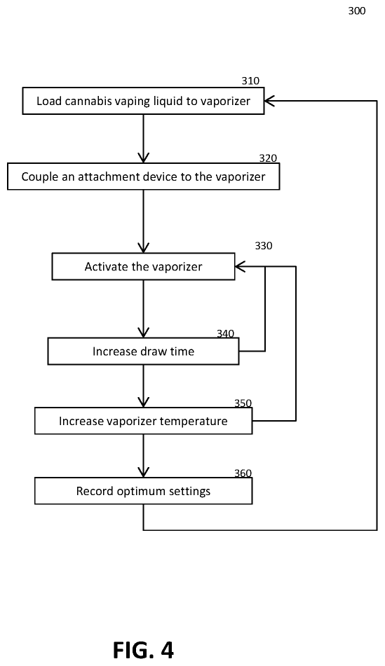

A method 300 for using the data acquisition module 132 of the attachment device to test various vaporizers and Cannabis-infused liquids is explained with reference to FIG. 4. FIG. 4 illustrates an exemplary method for obtaining data relating to dosage. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Testing the interactions between various vaporizers and various types of vaping liquid is essential in order to provide end users with an accurate analysis regarding the products they maybe use. The method 300 can begin at block 310, where a Cannabis-infused vaping liquid is loaded into a vaporizer. At block 320, the vaporizer is coupled with an attachment device. As described above, the attachment device can be a testing device. Information relating to the vaporizer being tested and the type of vaping liquid can be stored on a data acquisition module of the attachment device, as described with respect to FIG. 1. At block 330, the vaporizer can be activated to draw vaporized Cannabis liquid into the attachment device for a first draw period. In at least one example, the first draw period can correlate to the lowest temperature setting and the lowest activation time, shortest ‘puff’, the vaporize will allow. Data including, but not limited to, the amount of vaporized Cannabis liquid in the ‘puff,’ the potency of the vaporized Cannabis liquid in the ‘puff,’ and the amount of VOCs are recorded in the data acquisition module. At block 340, the vaporizer is activated to take a second draw wherein the draw period is increased incrementally from the first draw. In at least one example, the increase in draw period can be performed in intervals of one second. The method 300 can then repeat to the testing process to determine VOCs at each draw period increment. Once each draw increment has been tested, the method can proceed to block 350 where the temperature of the vaporizer is increased in incremental intervals, such as one degree Celsius, while the draw time is also incremented as described above. The method 300 can repeat the testing process until the vaporizer and Cannabis-infused vaping liquid combination has been tested at all possible temperatures and draw periods for which the vaporizer is operable. The method 300 can then proceed to block 360, where the data is saved to the data acquisition module of the attachment device.

The method 300 can be repeated for a plurality of vaporizer devices and Cannabis-infused vaping liquids. In at least one example, the method 300 can be repeated for each of the Cannabis-infused vaping liquids and each vaporizer available to the tester. The data relating to the vaping liquids, vaporizers, and the correlation between them can be accessible to users via a mobile device, as described with respect to FIG. 1, such that users are able to better understand the products they intend to use including the Cannabis-infused vaping liquids and the vaporizer device. In at least one example, the testing method 300 described herein can be performed by a Cannabis vaping liquid supplier, such that the information gathered could be included on their Cannabis vaping liquid packaging.

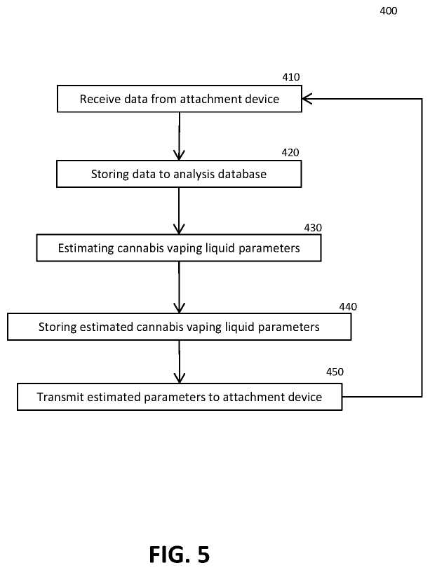

The functioning of the analysis module as described in FIG. 1. is explained with reference to FIG. 5, which illustrates an exemplary method for analyzing optimal settings for a vaporizer. The method 400 illustrates an exemplary interaction between the attachment device and the analysis network described with respect to FIG. 1. Specifically, the method 400 can begin at block 410 where the data acquisition module transmits sensor data stored in a memory of the attachment device to the analysis module of the analysis network. The data received at the analysis module can include, but is not limited to, the vaporizer and Cannabis-infused vaporizing liquid being used. In at least one example, the data can be received from the attachment device when a new measurement is detected at the attachment device. At block 420, the analysis module can store the received data in an analysis database maintained on the analysis network. At block 430, the analysis module can estimate parameters relating to the vaporized Cannabis vaping liquid and the optimal settings for the vaporizer being used. In at least one example, an analysis of the VOCs in the vapor can be measured in the draw chamber by means of a VOC sensor, such as a Sensirion Multi-Pixel Gas Sensor (SGP). Additional data analyzed can include, but is not limited to, the temperature, time, and TVOC content for a given vaporizer. As such, the optimal settings for the vaporizer can be estimated to allow for the highest temperature and draw possible while avoiding toxic effects. At block 440, the estimated parameters and the optimum settings can be stored in the analysis database of the analysis network. At block 450, the estimated parameters and optimum settings can be transmitted from the analysis network to the GUI of the attachment device via a communication network as described above. The end user of the Cannabis-infused vaping liquid can then access the estimated parameters either on the GUI of the attachment device or via the analysis network as described above. The end user can review the test results, to assist in determining the optimum settings to use for the specific vaporizer and Cannabis-infused vaping liquid pairing. The method can be repeated for any number of combinations of vaporizer devices and cannabis vaping liquids.

An exemplary entry from the analysis database of the analysis network is provided with respect to Table 1, below. It should be understood that the information provided with respect to Table 1 is merely exemplary and is not intended to limit the description in any way. It should be further understood that additional sensor data can be added without departing from the scope of the present disclosure.

TABLE 1

| | | | | | | | | | Estimated |
|---|---|---|---|---|---|---|---|---|---|
| | | | | | | Optimum | Optimum | Estimated | optimal |
| | Vaporizer | Vaping | Temp | Draw | TVOC | vaporizer | draw | optimal | draw |
| Vaporizer | ID | Liquid ID | (C.) | time(s) | (mg/m3) | setting | time(s) | temp(C.) | time(s) |
| Namaste | 001 | E101B001 | 190 | 3 | 0.15 | 191 | 4 | 195 | 3 |
| Namaste | 001 | E101B001 | 190 | 4 | 0.15 | . . . | . . . | . . . | . . . |
| Namaste | 001 | E101B001 | 190 | 5 | 0.18 | . . . | . . . | . . . | . . . |
| Namaste | 001 | E101B001 | 190 | 6 | 0.32 | . . . | . . . | . . . | . . . |
| Namaste | 001 | E101B001 | 191 | 3 | 0.18 | . . . | . . . | . . . | . . . |
| Namaste | 001 | E101B001 | 191 | 4 | 2.5 | . . . | . . . | . . . | . . . |
| Namaste | 001 | E101B001 | 191 | 5 | 12.3 | . . . | . . . | . . . | . . . |
| Namaste | 001 | E101B001 | 191 | 6 | 25 | . . . | . . . | . . . | . . . |

Table 1 illustrates an analysis database entry storing information including, but not limited to, vaporizer type information, vaping liquid ID, temperature ranges, draw times, and VOC measurements for various temperatures and draw times. Each table entry can be used to estimate optimal vaporizer settings. As shown, the analysis database can include estimated optimal vaporizer settings and/or vaping liquid parameters for vaping liquids and vaporizers which have not been tested using the device described herein. While all the data provided with respect to Table 1 correlates to a single vaporizer ID and vaping liquid ID, it should be understood that analysis database can include information relating to any number of vaporizers, oils, liquids, and combinations thereof.

In at least one example, the information gathered as a result of methods 300 and 400, described above, can be used to verify compliance with safety regulations. For example, the testing performed on the vaporizer can be used to determine the amount of vaporized cannabis liquid is provided to the user. Additionally, the attachment device can be used to verify that user has the vaping liquid that they believed the purchased. For example, the attachment device can run an analysis on the vaping liquid and verify that it correlates to information saved in the analysis database. As such, the methods and systems described herein can be used to ensure the end user has the product they intended to use and ensures they are only receiving a desired amount.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for providing dosage quality control information, the method comprising:
drawing vaporized Cannabis vaping liquid from a vaporizer device into a draw chamber over a plurality of draw periods;
detecting a plurality of parameters relating to the vaporized Cannabis vaping liquid via one or more sensors for each of the plurality of draw periods, the plurality of parameters for each draw period including at least one of a plurality of different temperatures;
transmitting the detected plurality of parameters for each of the draw periods over a communication network to an analysis network server;
receiving a plurality of analyses, each of the plurality of analyses based on the detected parameters for one of the plurality of draw periods and sent from the analysis network server over the communication network;
storing the plurality of analyses on an analysis database of the analysis network server; and
generating a display based on the plurality of analyses, wherein the display is presented via a graphic user interface.

2. The method of claim 1, wherein the sensors includes at least one volatile organic compound (VOC) sensor that detects one or more VOCs present in the vaporized Cannabis vaping liquid, and wherein the analysis of the detected parameters includes at least one of a volatile organic compounds (VOCs) assessment, a cannabidiol content (CBD), a delta-9-tetrahydrocannabinol (THC) content, a solvent content, and a diluent content.

3. The method of claim 1, further comprising transmitting information regarding the vaporizer device to the analysis network server, the transmitted information including at least one of a vaporizer model, an operation temperature range, and a draw cycle range.

4. The method of claim 1, further comprising transmitting information regarding the cannabis vaping liquid to the analysis network server, the transmitted information including at least one of cannabinoid content, a solvent, and a diluent.

5. The method of claim 1, wherein the plurality of analyses are available to be retrieved over the communication network.

6. The method of claim 1, further comprising tracking a plurality of draws of the vaporized Cannabis vaping liquid at the vaporizer device over time.

7. The method of claim 1, further comprising tracking a plurality of releases of the vaporized Cannabis vaping liquid at the vaporizer device over time.

8. The method of claim 1, further comprising making adjustments to a temperature at which the vaporizer device vaporizes the Cannabis vaping liquid.

9. The method of claim 8, wherein the analysis includes one or more recommended settings for the vaporizer device.

10. A device for providing dosage quality control information, the device comprising:
a draw chamber that draws in vaporized Cannabis vaping liquid from a vaporizer device over a plurality of draw periods;
one or more sensors that detect a plurality of parameters relating to the vaporized Cannabis vaping liquid at each of the plurality of draw periods, wherein the plurality of parameters for each draw period including at least one of a plurality of temperatures;
a communication network interface that:
transmits the detected plurality of parameters over a communication network to an analysis network server,
receives a plurality of analyses, each of the plurality of analyses based on the detected plurality of parameters for one of the plurality of draw periods and sent from the analysis network server over the communication network,
stores the plurality of analyses on an analysis database of the analysis network server; and
a graphic user interface that presents a display generated based on the received plurality of analyses.

11. The device of claim 10, wherein the sensors includes at least one volatile organic compound (VOC) sensor that detects one or more VOCs present in the vaporized Cannabis vaping liquid, and wherein the analysis of the detected parameters includes at least one of a volatile organic compounds (VOCs) assessment, a cannabidiol content (CBD), a delta-9-tetrahydrocannabinol (THC) content, a solvent content, and a diluent content.

12. The device of claim 10, wherein the communication network interface further transmits information regarding the vaporizer device to the analysis network server, the transmitted information including at least one of a vaporizer model, an operation temperature range, and a draw cycle range.

13. The device of claim 10, wherein the communication network interface further transmits information regarding the Cannabis vaping liquid to the analysis network server, the transmitted information including at least one of cannabinoid content, a solvent, and a diluent.

14. The device of claim 10, wherein the plurality of analyses are available to be retrieved over the communication network.

15. The device of claim 10, wherein the sensors further track a plurality of draws of the vaporized Cannabis vaping liquid at the vaporizer device over time.

16. The device of claim 10, wherein the sensors further track a plurality of releases of the vaporized Cannabis vaping liquid at the vaporizer device over time.

17. The device of claim 10, further comprising a controller that makes adjustments to a temperature at which the vaporizer device vaporizes the Cannabis vaping liquid.

18. The device of claim 17, wherein the analysis includes one or more recommended settings for the vaporizer device.
